(19) United States
(12) Patent Application Publication
(10) Pub. No.: US 2015/0190418 A1
(43) Pub. Date: Jul. 9, 2015

(54) COMBINATION PREPARATIONS OF 3-N-FORMYL HYDROXY AMINO PROPYL PHOSPHONIC ACID DERIVATIVES OR 3-N-ACETYL HYDROXY AMINO PROPYL PHOSPHONIC ACID DERIVATIVES TOGETHER WITH SPECIAL PHARMACEUTICAL ACTIVE INGREDIENTS

(71) Applicant: Jomaa Pharmaka GmbH, Giessen (DE)

(72) Inventors: Hassan Jomaa, Giessen (DE); Jochen Wiesner, Giessen (DE)

(73) Assignee: Jomaa Pharmaka GmbH, Giessen (DE)

(21) Appl. No.: 14/659,454

(22) Filed: Mar. 16, 2015

Related U.S. Application Data
(63) Continuation of application No. 10/363,280, filed on Oct. 1, 2003, now abandoned.

(30) Foreign Application Priority Data
Jun. 29, 2000 (DE) 10030781.7

(51) Int. Cl. A61K 31/7056; A61K 31/662

(52) U.S. Cl. CPC A61K 31/7056; A61K 31/662

(57) ABSTRACT

The invention relates to pharmaceutical combination preparations for use in the therapeutical and prophylactic treatment of bacterial and parasitic infections, especially malaria. The inventive preparations contain as the active substances 3-N-formyl-hydroxylaminopropyl phosphonic acid derivatives or 3-N-acetylhydroxylaminopropyl phosphonic acid derivatives combined with other pharmaceutical active agents.

The present invention relates to pharmaceutical preparations comprising 3-N-formyl hydroxy amino propyl phosphonic acid derivatives or 3-N-acetyl hydroxy amino propyl phosphonic acid derivatives as active ingredients in combination with special pharmaceutical active ingredients.

The use of 3-N-formyl hydroxy amino propyl phosphonic acid derivatives and 3-N-acetyl hydroxy amino propyl phosphonic acid derivatives for prophylactic and therapeutic treatment of infectious processes, especially infections caused by unicellular parasites (with the meaning of this invention: protozoa) or multicellular parasites, is already known from DE-A1-198 25 585. A bacterial activity has already been described in DE-A1-27 23 658. Even if these compounds exhibit good results in the treatment of infections caused by parasites or bacteria, also these medicaments exhibit undesired side-effects.

Therefore, the present invention made it its' object to enhance activity of these pharmaceutical preparations without increasing the side-effects of these active ingredients. Pharmaceutical preparations shall be made available providing a reduction of side-effects. The object is as well to widen the range of therapeutic application of said pharmaceutical preparations and especially also to extend it to the treatment of problematic groups such as children, and pregnant women. The antiparasitic activity shall be increased to such a degree such that these pharmaceutical preparations may be administered in lower doses and, thus, a reduction or elimination of side effects caused by these preparations is achieved.

Surprisingly, it has been found that 3-N-formyl hydroxy amino propyl phosphonic acid derivatives and 3-N-acetyl hydroxy amino propyl phosphonic acid derivatives in combination with a further pharmaceutical preparation being selected from the group consisting of clindamycin, lincomycin, mirincamycin, pirlimycin and other lincosamides; minocycline and other tetracycline derivatives, azithromycin, erythromycin, spiramycin, josamycin, roxithromycin, clarithromycin, midecamycin and other macrolide antibiotics, tiamuline, rifampicin, clotrimazole, flutrimazole, ketoconazole, tebuconazole, miconazole, itraconazole, fluconazole and other azole antimycotics; ciprofloxacin, norfloxacin, ofloxacin and other inhibitors of prokaryotic gyrase, nitrofurantoin, ornidazole, tinidazole, nimorazole and other nitroimidazole derivatives; disulfiram and other dithiocarbamates; lumefantrine, tafenoquine (WR238,605), pyronaridine, dihydroartemisinin, artemether, arteether, artesunate, isoniazid, chlorproguanil, trimethoprim and tetroxoprim, a significant higher therapeutic efficiency than in monotherapy. These combination preparations are especially suited for treatment of Malaria.

According to the present invention 3-N-formyl-hydroxy amino propyl phosphonic acid derivatives and 3-N-acetyl hydroxy amino propyl phosphonic acid derivatives are deemed to be compounds of formula (I)

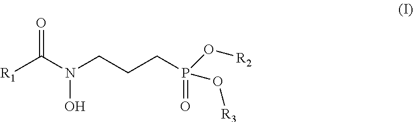

wherein R₁ is selected from the group consisting of hydrogen and methyl,
and
wherein R₂ and R₃ are independently selected from the group, consisting of hydrogen, substituted or unsubstituted alkyl, substituted or unsubstituted acyl, substituted or unsubstituted aryl, substituted or unsubstituted aralkyl, substituted or unsubstituted cycloalkyl, substituted or unsubstituted silyl, substituted or unsubstituted heterocyclic residue, or together form a substituted or unsubstituted C₁₋₅-alkyl chain, the alkyl groups being saturated or comprising one or more double bonds or triple bonds.

Lumefantrine, tafenoquine (WR238,605), pyronaridine, dihydroartemisinin, artemether, arteether, artesunate, clindamycin and azithromycin are especially preferred for the second pharmaceutical agent in the treatment of parasitic infections.

Clindamycin and azithromycin are especially preferred in the treatment of bacterial as well as parasitic infections. The combination preparation of clindamycin or azithromycin is especially suited for the treatment of infections caused by Helicobacter pylori.

The combination preparations are also deemed to be the respective salts, such as especially a fosmidomycin clindamycin salt and salts of ocher lincosamides.

Special features of the above definitions and suitable examples thereof are stated below:
“Acyl” is a substituent which originates from an acid, such as from an organic carboxylic acid, carbonic acid, carbamic acid or the thioacid or imidic acid corresponding to the individual above-stated acids, or from an organic sulfonic acid, wherein these acids may in each case comprise aliphatic, aromatic and/or heterocyclic groups in the molecule, as well as carbamoyl or carbamimidoyl.

Suitable examples of these acyl groups are stated below.

Aliphatic acyl croups are deemed to comprise acyl residues originating from an aliphatic acid, such groups including the following:
alkenoyl (for example formyl, acetyl, propionyl, butyryl, isobutyryl, valeryl, isovaleryl, pivaloyl etc.);
alkenoyl (for example acryloyl, methacryloyl, crotonoyl etc.);
alkylthioalkanoyl (for example methylthioacetyl, ethylthioacetyl etc.);
alkanesulfonyl (for example mesyl, ethanesulfonyl, propanesulfonyl etc.);
alkoxycarbonyl (for example methoxycarbonyl, ethoxycarbonyl, propoxycarbonyl, isopropoxycarbonyl, butoxycarbonyl, isobutoxycarbonyl etc.);
alkylcarbamoyl (for example methylcarbamoyl etc.);
(N-alkyl)thiocarbamoyl (for example (N-methyl)thiocarbamoyl etc.);
alkylcarbamimidoyl (for example methylcarbamimidoyl etc.); oxalo;
alkoxalyl (for example methoxalyl, ethoxalyl, propoxalyl etc.).

In the above examples of aliphatic acyl groups, the aliphatic hydrocarbon moiety, in particular the alkyl group or alkane residue, may optionally comprise one or more suitable substituents, such as amino, halogen (for example fluorine, chlorine, bromine etc.), hydroxy, hydroxyimino, carboxy, alkoxy (for example methoxy, ethoxy, propoxy etc.), alkoxycarbonyl, acylamino (for example benzyloxycarbonylamino etc.), acyloxy (for example acetoxy, benzyloxy etc.) and the like; preferred aliphatic acyl residues having such substituents which may be mentioned are alkanoyls substituted, for example, with amino, carboxy, amino and carboxy, halogen, acylamino or the like.

Aromatic acyl residues are deemed to comprise those acyl residue which originate from an acid with a substituted or unsubstituted aryl group, wherein the aryl group may comprise phenyl, toluyl, xylyl, naphthyl and the like; suitable examples are stated below:
aroyl (for example benzoyl, toluoyl, xyloyl, naphthoyl, phthaloyl etc.);
aralkanoyl (for example phenylacetyl etc.);
aralkanoyl (for example cinnamoyl etc.);
aryloxyalkanoyl (for example phenoxyacetyl etc.);
arylthioalkanoyl (for example phenylthioacetyl etc.);
arylaminoalkanoyl (for example N-phenylglycyl etc.);
arenesulfonyl (for example benzenesulfonyl, tosyl or toluenesulfonyl, naphthalenesulfonyl etc.);
aryloxycarbonyl (for example phenoxycarbonyl, naphthyloxycarbonyl etc.);
aralkoxycarbonyl (for example benzyloxycarbonyl etc.);
arylcarbamoyl (for example phenylcarbamoyl, naphthylcarbamoyl etc.);
arylglyoxyloyl (for example phenylglyoxyloyl etc.).

In the above examples of acyl residues, the aromatic hydrocarbon moiety (in particular the aryl residue) and/or the aliphatic hydrocarbon moiety (in particular the alkane residue) may optionally comprise one or more suitable substituents, such as those which have already been stated as suitable substituents for the alkyl group or the alkane residue. Aromatic acyl residues having particular substituents which may in particular be mentioned and constitute examples of preferred aromatic acyl residues are aroyl substituted with halogen and hydroxy or with halogen and acyloxy, and aralkanoyl substituted with hydroxy, hydroxyimino, dihaloalkanoyloxyimino, together with
arylthiocarbamoyl (for example phenylthiocarbamoyl etc.);
arylcarbamimidoyl (for example phenylcarbamimidoyl etc.).

A heterocyclic acyl residue is taken to mean an acyl residue which originates from an acid with a heterocyclic group; these include:
heterocyclic carbonyl, wherein the heterocyclic residue is an aromatic or aliphatic 5- to 6-membered heterocycle with at least one heteroatom from the group comprising nitrogen, oxygen and sulphur (for example thiophenyl, furoyl, pyrrolocarbonyl, nicotinoyl etc.);
alkanoyl heterocycle, wherein the heterocyclic residue is 5- to 6-membered and comprises at least one heteroatom from the group comprising nitrogen, oxygen and sulphur (for example thiophenylacetyl, furylacetyl, imidazolylpropionyl, tetrazolylacetyl, 2-(2-amino-4-thiazolyl)-2-methoxyiminoacetyl etc.) and the like.

In the above examples of heterocyclic acyl residues, the heterocycle and/or the aliphatic hydrocarbon moiety may optionally comprise one or more suitable substituents, such as those as have been stated to be suitable for alkyl and alkane groups.

“Alkyl groups” are straight- or branched-chain alkyl residues having 1 to 24 carbon atoms, such as methyl, ethyl, propyl, isopropyl, butyl, isobutyl, tert.-butyl, pentyl, hexyl and the like. They may be e.g. substituted with hydroxy, halogen or oxy groups.

Cycloalkyl preferably represents a optionally substituted C₃₋₈-cycloalkyl; a. o. alkoxy (e.g. methoxy, ethoxy, etc.), halogen (e.g. fluorine, chlorine, bromine etc.), nitro and the like are suited to be possible substituents.

Aryl is an aromatic hydrocarbon residue, such as phenyl, naphthyl etc., which may optionally comprise one or more suitable substituents such as alkyl, alkoxy (for example methoxy, ethoxy etc.), trifluoromethylene, halogen (for example fluorine, chlorine, bromine etc.), nitro and the like.

“Aralkyl” includes mono-, di-, triphenylalkyl such as benzyl, phenethyl, benzhydryl, trityl and the like wherein the aromatic moiety may optionally comprise one or more suitable substituents such as alkoxy (for example methoxy, ethoxy etc.), halogen (for example fluorine, chlorine, bromine etc.), nitro and the like.

In the above ester the alkane and/or arene moiety may optionally comprise at least one suitable substituent, such as halogen, alkoxy, hydroxy, nitro and the like.

The invention further relates to the use of 3-N-formyl hydroxy amino propyl phosphonic acid derivatives or 3-N-acetyl hydroxy amino propyl phosphonic acid derivatives in combination with clindamycin, lincomycin, mirincamycin, pirlimycin and other lincosamides, minocycline and other tetracycline derivatives, azithromycin, erythromycin, spiramycin, josamycin, roxithromycin, clarithromycin, midecamycin and other macrolide antibiotics, tiamuline, rifampicin, clotrimazole, flutrimazole, ketoconazole, tebuconazole, miconazole, itraconazole, fluconazole and other azole antimycotics, ciprofloxacin, norfloxacin, ofloxacin and other inhibitors of prokaryotic gyrase, nitrofurantoin, ornidazole, tinidazole, nimorazole and other nitroimidazole derivatives, disulfiram and ocher dithiocarbamates, lumefantrine, tafenoquine (WR238,605), pyronaridine, dihydroartemisinin, artemether, arteether, artesunate, isoniazid, chlorproguanil, trimethoprim and tetroxoprim for therapeutic and prophylactic treatment of infections caused by bacteria, protozoa or multicellular parasites.

The use of combination therapy with the help of pharmaceutical preparations of the present invention has the advantage of a synergistic increase of antiparasitic activity of the single substances. Hence, in combining the single compounds, there is a possibility of reducing the doses and, thus, the toxicity of the single compounds at the same time preserving antiparasitic activity. A combination therapy of the above listed principles of therapy of the individual compounds further provides the possibility of overcoming resistance.

With the use of said combination therapy it is possible to administer minister the active agents in a so-called fixed combination, i.e. in a single pharmaceutical formulation containing both the active agents or to choose a so-called free combination, administering the active agents in form of separate pharmaceutical formulations at the same time or one after the other.

If the active agents are solid materials, the active agents may be administered by conventional methods for solid drug preparations mixing e.g. both active agents and pelletizing them, for example into pellets together with conventional excipients or auxiliary materials. However, it is also possible to provide the active agents separately in one package unit ready for sale wherein the package unit contains both active agents in separate pharmaceutical formulations.

The pharmaceutical preparations may be administered in liquid or solid form for enteral or parenteral application. In this connection all conventional forms of application are possible, for example pellets, capsules, dragees, sirups, solutions, suspensions. Preferably, water is used as an injection medium containing added substances common in injection solutions such as stabilizers, dissolving intermediaries and buffers. If desired, preparations suited for oral application may contain flavorings or sweeteners.

The following example states the favourable activity of some representative combination preparations.

### EXAMPLE

The sensitivity of Plasmodium falciparum in view of fosmidomycin in combination with different compounds has been determined in a semi-automatic test system by the incorporation of (³H)-hypoxanthin into the DNA of parasites. The IC50-values of fosmidomycin and the respective combination partner were determined for the single compounds and in different ratios of mixture on microtitreplates. The results were defined as sum of fractional inhibitory concentration (sum fractional inhibitory concentration, FIC):

sum FIC=IC50 of fosmidomycin in mixture/IC50 of fosmidomycin alone+IC 50 of the combination partners in mixture/IC50 of combination partner alone

Sum FIC-values<1 represent synergism, values>1 antargonism and values=1 addition. It has to be considered that also slightly antargonistic combinations may be therapeutically valuable (sum FIC<2), because both drugs need not to be administered in full doses necessary for monotherapy. In this case, the advantageous effect is the particularly quick killing of parasites and the avoidance of resistance.

The following sum FIC-values have been measured on Plasmodium falciparum strains Dd2, 3D7 and HB3:

| drug | P. falciparum strain | sum FIC |
|---|---|---|
| clindamycin | Dd2 | 0.42 |
| | 3D7 | 0.40 |
| | HB3 | 0.37 |
| azithromycin | Dd2 | 0.86 |
| | 3D7 | 0.74 |
| | HB3 | 0.85 |
| lumefantrin | Dd2 | 1.20 |
| | HB3 | 1.08 |
| | 3D7 | 1.21 |

1-7. (canceled)

8. A pharmaceutical composition comprising synergistic effective amounts of fosmidomycin and clindamycin, and a pharmaceutically acceptable carrier.

9. A method of treating malaria comprising: administering to a mammal in need of treatment of malaria synergistic effective amounts of fosmidomycin and clindamycin.

10. The method of claim 9, wherein fosmidomycin and clindamycin are administered at the same time.

11. The method of claim 9, wherein fosmidomycin and clindamycin are administered sequentially.

12. The method of claim 9, wherein fosmidomycin and clindamycin are administered orally, enterally, or parenterally.
